(19) United States
(12) Patent Application Publication
(10) Pub. No.: US 2010/0247685 A1
(43) Pub. Date: Sep. 30, 2010

(54) Methods For Treating and Reducing the Incidence of Newcastle Disease

(75) Inventors: Richard Rosenbloom, North Wales, PA (US); Timothy Cummings, Mississippi State, MS (US); Michael Petteruti, Hilltown, PA (US)

Correspondence Address: KNOBLE, YOSHIDA & DUNLEAVY, EIGHT PENN CENTER, SUITE 1350, 1628 JOHN F KENNEDY BLVD, PHILADELPHIA, PA, 19103, US

(73) Assignee: THE QUIGLEY CORPORATION, Doylestown, PA (US)

(21) Appl. No.: 12/513,987

(22) PCT Filed: Dec. 14, 2007
(86) PCT No.: PCT/US07/87536

§ 371 Date: May 7, 2009

Related U.S. Application Data
(60) Provisional application No. 60/869,987, filed on Dec. 14, 2006.

(51) Int. Cl. A61K 36/82; A61P 31/12

(52) U.S. Cl. 424/729

(57) ABSTRACT

A prophylactic treatment for Newcastle disease as well as a treatment of Newcastle disease. The methods comprise the step of administering to a bird, an amount of a composition having a first ingredient obtainable from turmeric; a second ingredient obtainable from green tea; an third ingredient obtainable from ginger; and an acceptable carrier. Compositions in accordance with the invention may be employed for the purpose of reducing the incidence of contracting Newcastle disease or reducing the transmissivity of Newcastle disease.

### BACKGROUND OF THE INVENTION

1. Field of the Invention

The present invention relates methods for treating or reducing the incidence of Newcastle disease. More particularly, the present invention relates to methods for treating, or reducing the incidence of Newcastle disease in birds as well as reducing the transmissivity of Newcastle disease.

2. Description of the Related Technology

Newcastle disease is an acute, febrile and highly contagious disease that affects the respiratory and nervous systems of birds. Highly contagious and easily transmitted, the disease has been the cause of numerous epidemics in birds since 1926. The disease generally appears suddenly and spreads quickly in susceptible flocks through direct contact with the secretions, feces, respiratory discharges and carcass, frozen or otherwise, of infected fowl or previously vaccinated fowl. The disease is also transmitted by contaminated surfaces, such as feed, water, implements or premises. The disease may result in asymptomatic infection to heavy mortality. In younger birds, mortality may range from a low percentage to 100%. Similarly, mortality in laying flocks varies from 0 to 100% depending on the viral strain. To date, there is no known treatment or cure for Newcastle disease.

Newcastle disease is a paramyxovirus belonging to the group of myxoviruses and has many different viral strains and forms. All strains of Newcastle disease are morphologically, structurally and serologically indistinguishable. However, large differences exist in the virulence of different strains for chickens, eggs and tissue culture systems. These differences are expressed in the classification of the different strains as velogenic, mesogenic and lentogenic strains.

Lentogenic strains, apathogenic, are used in the preparations of most live vaccines. Lentogenic Newcastle disease is caused by mildly pathogenic strains of Newcastle disease virus and is characterized mainly by respiratory problems. This form of the disease is commonplace in the commercial poultry industry and results in economic losses due to livestock loss, poor livestock growth, feed conversion, and increased livestock carcass condemnation at processing.

Mesogenic strains, which is intermediate in pathogenicity, are used as vaccinal strains for boosting the immunity of older fowls, e.g. in such commercially available mesogenic strains as Komarov (Haifa) and Roakin. Mesogenic Newcastle disease is typically found throughout the world in various fowl.

Velogenic strains are highly pathogenic and are used to test immunity and mortality. Velogenic viscerotropic Newcastle disease is characterized by high mortality with severe lesions in the gastrointestinal tract. Although present in most other areas of the world, it is not found in the United States. The last outbreak of velogenic viscerotropic Newcastle disease occurred in California during 1970-74, costing the United States Department of Agriculture (USDA) more than 60 million dollars to eradicate the disease. Velogenic neurotropic Newcastle disease is also characterized by a high mortality and severe neurological symptoms. Although seldom seen in the United States, Velogenic neurotropic Newcastle disease is relatively widespread in other parts of the world.

In an effort to curtail the economic losses due to Newcastle disease in the commercial poultry industry, young chickens have been routinely vaccinated against the Newcastle disease virus. The vaccines used are prepared with live attenuated Newcastle disease virus derived from lentogenic or mesogenic strains and confer immunity against all forms of Newcastle disease.

Chickens are typically inoculated by mass administration procedures including the distribution of the live virus vaccine in drinking water and the spraying of the vaccine directly onto the chickens. Once inside the chicken, the virus replicates in the respiratory tract and is spread from chicken to chicken by aerosol and direct contact routes, theoretically immunizing a flock within to the various forms of Newcastle disease within a relatively short amount of time. This immunization, however, frequently induces a subclinical or mild clinical form of Newcastle disease. Any conferred immunity tends to be non-uniform among the flock and of short duration. Inactivated vaccines are also inadequate, requiring the use of large amounts of antigen to induce immunity that is inefficiently administered parenterally and may contain allergenic substances, i.e. antibiotics, preservatives, etc. Moreover, because the immune response increases as the pathogenicity of the live vaccine increases, immunization programs often involve sequential administration of multiple and progressively more virulent viruses or live virus followed by administration of vaccines using inactivated viruses. Another significant problem is that some virulent strains, such as the exotic Newcastle disease strain, are capable of infecting and killing birds that have been previously vaccinated.

As a result of these common problems, investigations have been conducted to develop non-virus based treatment methods. Although there are some publications, such as U.S. publications nos. 2003/0185912 and 2005/0147697, that address the antiviral properties of a composition composed of ginger, green tea, turmeric and horseradish, such publications do not disclose a method for treating Newcastle disease.

Therefore, a need exists for methods of treating, reducing the incidence of, or reducing the transmissmivity of Newcastle disease.

### BRIEF DESCRIPTION OF THE DRAWINGS

FIG. 1 shows a graph of dilution log versus cell concentration depicting the affect of Composition 1 on the viability of Newcastle disease virus (NDV) strain B1/B1 in VERO E6 cells

FIG. 2 shows a graph of dilution log versus cell concentration depicting the affect of a composition in accordance with the invention on the viability of NDV strain B1/B1 in embryonating eggs.

FIG. 3 shows on the left panel, cells showing no cytopathic effects (CPE) following exposure to NDV treated with a 1×10⁻³ dilution of a composition in accordance with the present invention, and on the right panel, cells with CPE following exposure to NDV treated with a 1×10⁻³ dilution of the placebo.

### SUMMARY OF THE INVENTION

The invention is directed to methods for the treatment of Newcastle disease, for reducing the incidence of contracting Newcastle disease and for reducing the transmissivity of Newcastle disease.

In the first aspect of the invention, relates to a method for the prophylactic use of a composition to reduce the incidence of contracting and/or transmitting Newcastle disease. The method comprises the steps of administering to a bird that has been, might be or will be, exposed to the Newcastle virus, an amount of a composition having a first ingredient obtainable from turmeric extract; a second ingredient obtainable from green tea; and an acceptable carrier. The amount of anti-microbial composition is effective, when administered, to reduce the incidence of contracting and/or transmitting Newcastle disease.

In a second aspect of the invention, the composition may be used to treat a bird infected with Newcastle disease by administering an effective amount of the composition to treat Newcastle disease.

A third aspect of the invention relates to aerosol or liquid compositions including having a first ingredient obtainable from turmeric; a second ingredient obtainable from green tea; and an acceptable aerosol spray vehicle. The aerosol composition may be administered as a treatment or prophylactically to birds using any conventional techniques for which an aerosol, spray or mist composition is suitable, e.g. spraying or misting of birds.

In a fourth aspect, the present invention relates to animal feeds, dry formulations and liquid formulations which includes a first ingredient obtainable from turmeric and a second ingredient obtainable from green tea.

These and various other advantages and features of novelty that characterize the invention are pointed out with particularity in the claims annexed hereto and forming a part hereof. However, for a better understanding of the invention, its advantages, and the objects obtained by its use, reference should be made to the accompanying descriptive matter, in which there is described, a preferred embodiment of the invention.

### DETAILED DESCRIPTION OF THE PREFERRED EMBODIMENTS

The invention relates to a method for the prophylactic use of a composition to reduce the incidence of contracting and/or transmitting Newcastle disease. The method comprises the steps of administering to a bird that has been, or will be, exposed to the Newcastle virus, an amount of a composition having a first ingredient obtainable from turmeric; a second ingredient obtainable from green tea; and an acceptable carrier. The amount of anti-microbial composition is effective, when administered, to reduce the incidence of contracting and/or transmitting Newcastle disease. The composition may also be used to treat a bird infected with Newcastle disease by administering an effective amount of the composition to treat Newcastle disease.

The composition for use in the methods of the present invention may include a first ingredient obtainable from turmeric, and a second ingredient obtainable from green tea. Ingredients obtainable from ginger and horseradish may also be included as optional additional ingredients of the composition of the present invention.

As used herein, the term “acceptable” means a component that is suitable for use with birds without undue adverse side effects (such as toxicity, irritation, and allergic responses), commensurate with a reasonable risk/benefit ratio.

Further, as used herein, the term “safe and effective amount” refers to the quantity of a component, which is sufficient to yield a desired therapeutic response without undue adverse side effects (such as toxicity, irritation, or allergic responses), commensurate with a reasonable risk/benefit ratio when used in the manner described herein.

The term “inhibiting”, as used herein, refers to reducing or preventing further growth of a Newcastle virus strain, or preventing the Newcastle viral strain from attaching to normal cells, and/or the elimination of some or all of the infectious particles from the human or animal being treated. Suitable methods for determining viral inhibition are discussed in the examples.

The term “transmissivity” or “transmitting” as used herein refers to the transfer of a microbe from one host to another.

All active compounds used in the present invention may be obtained from other sources, if available. Thus, the phrase “which can be obtained from” or the phrase “which may be obtained from” is meant to encompass compounds or compositions that are obtainable from turmeric, ginger, green tea or horseradish, and therefore encompasses synthetic forms of the same compounds and/or compositions as well as the same compounds and/or compositions obtained from other sources.

In a first embodiment, the composition of the present invention includes a first ingredient obtainable from turmeric, and a second ingredient obtainable from green tea, in a safe and effective amount to provide one or more of the beneficial effects described herein.

The first ingredient of the composition of the present invention may be obtained from turmeric. The ingredient obtained from turmeric may be used in a safe and effective amount to provide one or more of the beneficial effects described herein. Turmeric (Curcuma longa), or Haldi in Hindi, is used very widely as medicine as well as a common ingredient in Indian cooking. The rhizome of turmeric is used in medicine and food as a fine powder.

The yellow pigment of the rhizome of turmeric is composed of three compounds known as curcuminoids. The three curcuminoids are curcumin (diferuloylmethane), desmethoxycurcumin (hydroxycinnamoyl feruloylmethane), and bis-desmethoxycurcumin (dihydroxydicinnamoyl methane) (see Drug Analysis, Chromatography and Microscopy, p. 169, Ann Arbor Science Inc., 1973). The essential oils of turmeric (Curcuma longa) are primarily composed of the following compounds: d-camphor (about 1%), cyclo-isoprenemyrcene (about 85%), and p-tolylmethylcarbinol (about 5%), (see E. Gunther, The Essential Oil, pp. 123-4, Van Nostrand Co., 1955).

The ingredient of the composition of the present invention, obtained from turmeric, preferably includes curcuminoids, such as curcumin (diferuloylmethane), desmethoxycurcumin (hydroxycinnamoyl feruloylmethane), and bis-desmethoxycurcumin (dihydroxydicinnamoyl methane), and mixtures of two or more of these curcuminoids.

Methods for isolating curcuminoids from turmeric are known (see Janaki and Bose, An Improved Method for the Isolation of Curcumin From Turmeric, J. Indian Chem. Soc. 44: 985, 1967). Alternatively, curcuminoids for use in the present invention can be prepared by synthetic methods.

The ingredient, which can be obtained from of turmeric, can be incorporated into the composition of the present invention in a variety of different forms. Those different forms preferably include extracts of turmeric such as turmeric extracts including turmeric powder extracts, turmeric fluid extracts, Aquaresin® turmeric, Oleoresin® turmeric, one or more the curcuminoid compounds, and turmeric powder, parts of, or whole plants of turmeric, tinctures thereof, and mixtures thereof. More preferably, the first ingredient obtainable from turmeric is a turmeric extract.

When the ingredient obtainable from turmeric is used, each gram of the composition of the present invention preferably contains about 0.001 mg to about 20 mg of an ingredient obtainable from turmeric such as turmeric powder extract. Most preferably, each gram of the compositions contains about 0.01 mg to about 1.5 mg of an ingredient obtainable from turmeric such as turmeric powder extract. These ranges are based on the use of Turmeric Extract 95%, ex. Pharmline, Inc. in the ingested formulation and Turmeric Root Extract (Oleoresin® Turmeric), ex. Kalsec, Inc., Kalamazoo, Mich., in the spray formulation.

The second ingredient of the composition of the present invention may be obtained from green tea. The second ingredient obtained from green tea may have an antioxidant effect. Green tea is the dried leaves and leaf buds of the shrub Camellia sinensis. It is mainly produced in China and Japan. Dried tea leaves are composed mainly of phytochemicals known as polyphenols (about 36%), principally flavonols (including catechins), flavonoids, and flavondiols. The leaves also contain plant alkaloids (about 4%), including caffeine, theobromine and theophylline.

The pharmacological activities of green tea are mainly due to its active compounds. The active compounds of green tea useful in the present invention include, but are not limited to, flavonols, catechins, flavonoids, flavondiols, plant alkaloids, caffeine, theobromine, theophylline, phenolic acids, proteins, carbohydrates, and minerals.

The second ingredient which may be obtained from green tea, can be included in the composition in the form of green tea powder, green tea extracts such as green tea powder extracts, green tea fluid extracts, and one or more active compounds of green tea, part of, or whole green tea plants, green tea leaves, tinctures thereof, or mixtures thereof. Preferably, the second ingredient of the composition of the present invention is selected from green tea leaves, green tea powder and green tea extract. More preferably, the second ingredient of the composition of the present invention is green tea extract.

Each gram of the composition of the present invention preferably contains about 0.001 mg to about 20 mg of an ingredient obtainable from green tea such as green tea extract. Most preferably, each gram of the composition contains about 0.01 mg to about 15 mg of an ingredient obtainable from green tea such as green tea extract. These ranges use, as a baseline, the use of Green Tea, ex. Stryker Botanics in the ingested formulation and Green Tea Extract, ex. Phytoway, Inc., ChangSha, P.R. China, in the spray formulation.

An optional ingredient of the composition of the present invention may be obtained from ginger, and is used in a safe and effective amount. Native to southern Asia, ginger is a 2- to 4-foot perennial that produces grass-like leaves up to a foot long and almost an inch wide. Ginger root, as it is called in the grocery store, actually consists of the underground stem of the plant, with its bark-like outer covering scraped off.

The active compounds of ginger which may be employed in the present invention include, but are not limited to, 1,8-cineole, 10-dehydrogingerdione, 10-gingerol, 6-gingerdione, 6-gingerol, 6-shogaol, 8-.beta.-17-epoxy-.lambda.-trans-12-ene-15,16-diol, 8-gingerol, 8-shogaol, 9-oxo-nerolidol, acetaldehyde, acetic acid, alanine, .alpha.-linolenic-acid, .alpha.-linolenic acid, .alpha.-phellandrene, .alpha.-piene, .alpha.-terpinene, .alpha.-terpineol, .alpha.-zingiberene, ar-curcumene, arginine, ascorbic acid, asparagine, .beta.-bisabolol, .beta.-carotene, .beta.-elemene, .beta.-eudesmol, .beta.-ionone, .beta.-myrcene, .beta.-phellandrene, .beta.-pinene, .beta.-selinene, .beta.-sesquiphellandrene, .beta.-sitosterol, .beta.-thujone, bornyl-acetate, boron, caffeic acid, calcium, camphene, camphor, capric acid, caprylic acid, capsaicin, caryophyllene, chavicol, chlorogenic acid, chromium, citral, citronellal, citronellal, cobalt, copper, cumene, curcumin, cystine, delphinidin, .delta.-cadinene, elemol, ethyl acetate, ethyl-myristate, farnesal, farnesene, ferulic acid, furfural, .gamma.-aminobutyric acid, .gamma.-terpinene, geranial, geraniol, geranyl-acetate, gingerenone, glutamic acid, glycine, hexahydrocurcumin, histidine, isogingerenone-B, isoleucine, kaempferol, lecithin, limonene, linoleic acid, magnesium, manganese, methionine, mufa, myrecene, myricetin, myristic acid, neral, nerol, nerolidol, niacin, nickel, oleic acid, oxalic acid, p-coumaric acid, p-cymene, p-hydroxy-benzoic acid, palmitic acid, pantothenic acid, paradol, patchoulic alcohol, phenylalanine, quercetin, riboflavin, selenium, shikimic-acid, terpinen-4-ol, thiamin, tryptophan, vanillic acid, vanillin, zinc, and zingerone. Also, mixtures of two or more of these active compounds may be employed.

Ginger, can be incorporated in the composition of the present invention in many different forms including extracts such as ginger extracts including ginger powder extracts, ginger fluid extracts, ginger powder including ginger root powder, Aquaresin® ginger, Oleoresin® ginger, and one or more active compounds of ginger, parts of, or whole ginger plants, tinctures thereof, and mixtures thereof. Preferably, the optional ingredient of the composition of the present invention is selected from ginger extract and ginger powder.

Each gram of the composition of the present invention preferably contains about 0.001 mg to about 30 mg of an ingredient obtainable from ginger such as ginger extract. Most preferably, each gram of the composition contains about 0.01 mg to about 20 mg of an ingredient obtainable from ginger such as ginger extract. These ranges use, as a baseline, the use of Ginger Root Powder, ex. Stryka Botanics in the ingested formulation and Ginger Extract K (Aquaresin® ginger), ex. Kalsec, Inc. of Kalamazoo, Mich. in the spray formulation.

The amounts of various ingredients are given herein in terms of one form of the ingredient, i.e. ginger extract. If that ingredient is present in another form, then the amount to be employed is that amount which will provide the same amount of the one or more active compounds as the amount of that ingredient given herein.

Also, the composition of the present invention may include one or more ingredients obtainable from horseradish, in a safe and effective amount to provide one or more of the beneficial effects described herein. The optional ingredient obtainable from horseradish may include extracts from the Cochlearia Armoracia and may be in the form of a horseradish extract, such as, for example, horseradish powder extracts, horseradish fluid extracts, and horseradish root extracts such as horseradish oil. Horseradish contains volatile oils that are similar to those found in mustard. These include glucosinolates (mustard oil glycosides), gluconasturtiin, and sinigrin, which yield allyl isothiocynate when broken down in the stomach. The compositions of the present invention preferably contain from about 0.0001 mg to 10 mg of an ingredient obtainable from horseradish such as horseradish oil per gram of the composition, and more preferably, from 0.001 mg to 5 mg of an ingredient obtainable from horseradish such as horseradish oil per gram of the composition.

Exemplary compositions of the present invention may include one or more ingredients obtainable from each of turmeric and green tea; one or more ingredients obtainable from each of turmeric, green tea and ginger; one or more ingredients obtainable from each of turmeric, green tea and horseradish; or one or more ingredients obtainable from each of turmeric, green tea, ginger and horseradish.

Ethanol, propylene glycol and glycerin and various combinations thereof, may be optionally included in the liquid compositions of the present invention, up to about 10 percent by weight of the total as an optional ingredients. Most preferably, up to about 10 percent per total weight ethanol is added as an optional ingredient. Even more preferable, 2.5 to 7 percent ethanol is added.

Preferably, the main ingredients described above, that may be derived from turmeric and green tea and, optionally, ginger and horseradish, make up from about 0.001 to about 90% by weight of the total composition. More preferably, the main ingredients will make up about 0.01 to about 20% by weight of the total composition. Most preferably, the main ingredients make up about 1 to about 10% by weight of the total composition

The non-carrier ingredients of the composition, including the ingredients obtainable from turmeric, green tea, ginger and horseradish as discussed above, can be increased or decreased proportionally in the composition of the present invention depending on the amount of carrier used in the composition, without substantially affecting the effectiveness of the composition for its intended use.

The plant extracts, e.g., turmeric extract, ginger extract, green tea extract and horseradish extract that may be used in the compositions of the invention, may be produced using common extraction procedures. Alternatively, the extracts may be purchased from commercial sources such as the Kalsec, Inc. of Kalamazoo, Mich.

The processes for the preparation of pharmacologically or biologically active plant extracts in a convenient, administrable dosage form from any of the plants mentioned above, are well known in the art.

The composition of the present invention may be used to prevent the infectivity and transmissivity of various strains of the Newcastle disease virus among birds. The composition may also be used as a therapeutic composition to treat Newcastle disease and symptoms associated with Newcastle disease.

A safe and effective amount of the composition of the present invention may be administered to a bird that has been or will be exposed to Newcastle disease, to reduce the incidence of contracting said illness, relative to a bird that has been or will be exposed to the Newcastle disease virus.

Preferably, the composition of the present invention may be formulated in any acceptable dosage form including, but not limited to animal feeds such as bird feed, powders, dry formulations of any type, liquid formulations of any type, such as sprays, suspensions, solutions, injections or any standard form for mass inoculation. The composition of the present invention may also be administered in the form of a nutritional supplement, in which case the composition of the invention may be the nutritional supplement or may form a part of a nutritional supplement containing additional ingredients.

Tablets in this invention may differ in shape, size and manufacturing technique. In the case of tablets, for oral use, the acceptable carrier may further include lactose and corn starch. Lubricating agents may also be added to the tablets, including, for example, magnesium stearate, sodium lauryl sulfate and talc. Tablets may also contain excipients such as sodium citrate, calcium carbonate and calcium phosphate. Disintegrants such as starch, alginic acid and complex silicates, may also be employed. Tablets may also include binding agents such as polyvinylpyrrolidone, gelatin, PEG-8000 and gum acacia.

Alternatively, the composition of the present invention may be formulated in liquid form, such as syrups, solutions, liquid formulations, mists or sprays, with a solvent or dispersant such as water, or other liquids and optionally in a pharmaceutically acceptable carrier, for repeated delivery of the composition to oral and oropharyngeal mucous membranes over a sustained period of time. Preferably, the treatment time is about 5 to 60 minutes, and more preferably about 20 to 30 minutes, so as to permit a prolonged contact of the composition with mouth, nasopharnyx and throat tissues. Alternatively, such formulations can be in a concentrated form suitable for dilution with water or other materials prior to use.

The composition of the present invention may also be formulated with an acceptable carrier. The acceptable carrier may include, but is not limited to: (a) glycerin; (b) ethanol; (c) phospholipids; (d) MCT oil; (e) water; and (f) suitable relatively insoluble excipients including starches, celluloses, cyclodextrins, silicas and lipids/fats.

The composition may also be formulated in chewable forms, such as animal feeds, as a food additive or component of the animal feed. The composition of the invention may alternatively be formulated in capsule form, with or without diluents. For capsules, useful diluents include lactose and dried corn starch. When suspensions are employed, emulsifying and/or suspending agents may be employed in the suspensions. In addition, solid compositions including one or more of the ingredients of the lozenges described above may be employed in soft and hard gelatin capsules.

The composition of the present invention may also be formulated into an aerosol or inhalant composition. Such a composition may be prepared using well-known techniques. For these types of formulations, suitable carriers may include the following ingredients: saline with one or more preservatives, absorption promoters to enhance bioavailability, fluorocarbons, and/or conventional solubilizing or dispersion agents.

The composition may also be administered using any known standard delivery means. The composition may be formulated as a water or solution additive. Moreover, the composition may be administered in ovo. Optionally, the compositions of the invention can also be used as an anti-viral disinfectant for wiping down surfaces.

Other materials, which may optionally be included in the composition of the present invention, include resveratrol (trihydroxystilbene), inositol, other B-complex vitamins, and additional anti-inflammatories. Also, ingredients such as sweeteners, flavorants, coloring agents, dyes, preservatives, emulsifying agents, suspending agents, melting agents, excipients, demulcents and solvents or diluents such as water, ethanol, propylene glycol, glycerin and various combinations thereof, may be included in the composition of the present invention.

Reducing or preventing transmission relates to preventing or reducing the spread of a microbe from one bird (infected) to another bird (non-infected). Some birds may be considered carriers of the infection. Carriers are individuals who actively shed Newcastle microbes but do not suffer from an acute infection. These carriers may be said to be persistently (or chronically) infected with a viral strain of Newcastle. In addition to the persistently infected shedder, other infective birds may be those which are actively infected, and particularly those in the early or late stages of an acute infection. One aspect of the invention relates to administering to a bird infected with a strain of Newcastle disease, the composition of the present invention, to prevent the spread of the disease to other birds.

Prophylactic treatment is aimed at a bird that will soon be exposed to the Newcastle virus or has recently been exposed to the Newcastle virus for the purpose of reducing the instance of active infection. Such prophylactic treatment may be effective either alone or in addition to a vaccine. The prophylactic treatment of the present invention may also be used against viral strains of Newcastle disease for which there is not yet a vaccine available.

The invention also relates to a method of treating a bird infected with Newcastle disease to treat the disease by, for example, reducing the duration, fatality rate, or adverse effects of the disease. In another aspect of the invention, the present invention relates to a method for reducing, treating or at least partially preventing of at least one symptom or adverse effect of viral infection by administering, to a bird infected with the Newcastle virus, a composition of the present invention, including ingredients that can be obtained from ginger and green tea. Symptoms that may be treated include lack of energy, decreased egg production, soft shelled eggs, swelling, nasal discharge, coughing, gasping, head tremors, wing and leg paralysis, twisted necks, impaired appetite, diarrhea, intestinal lesions, depression, loss of appetite, increased respiration and blue combs.

The composition of the present invention may be administered to any member of the avian species, which includes the common commercial poultry birds: chicken, turkeys, ducks and geese, less commonly the ostrich as well as other bird species that are commonly kept as house pets, for example canaries and parrots.

The composition may be administered by directly spraying the composition into the nasal passage of the bird, spraying the composition into the oral cavity of the bird or the composition may be administered by creating a mist to which the birds are exposed. Thus, the composition may be given prophylactically to act in a virucidal or virustatic manner. Alternatively, the composition may be used to reduce the transmissivity of the virus.

The effective amount of the composition will vary depending on such factors as the patient being treated, the particular mode of administration, the activity of the particular active ingredients employed, the age, bodyweight, general health, sex and diet of the bird, time of administration, rate of excretion, the particular combination of ingredients employed, the total content of the main ingredient of the composition, and the severity of the illness or symptom. It is within the purview of one of ordinary skill in the art to account for these factors.

The composition may be administered about 1 to about 15 times per day, as needed, more preferably, about 2 to about 12 times per day, as needed, or most preferably, about 6 to about 10 times per day, as needed. The composition of the present invention may be administered in any acceptable dosage form, as described above, including, but not limited to, tablets, capsules, powders, oral sprays, nasal sprays, nasal drops, chewable compositions, suspensions, solutions and through in ovo administration.

Each dosage of the composition contains a safe and effective amount of the composition of the present invention. An effective amount for each therapeutic administration contains a total of about 0.001 milligram to about 1 gram of the ingredients, which may be obtained from turmeric and green tea. More preferably, an effective amount of the composition for each therapeutic administration contains a total of about 0.01 milligrams to about 0.5 grams of the ingredients which may be obtained from turmeric and green tea. The amounts of the various ingredients of the composition administered in accordance with the method of the present invention are the same as given above for the composition of the present invention.

When the composition is administered as a feed or water additive, the amount of the active ingredients in the feed or water additive may range from about 0.01 to 50 weight percent of the total feed composition. In a preferred embodiment, the active ingredients constitute about 0.1 to about 30 weight percent of the total feed composition, and in a most preferred embodiment, the active ingredients constitute about 1 to about 20 weight percent of the total feed composition. The active ingredients may comprise the ingredient from turmeric, the ingredient from green tea, the ingredient from ginger and/or the ingredient from horseradish.

When the composition is administered as a liquid, mist, spray, injection, aerosol or mist, the amounts each of the active ingredients may be reduced as the spray composition delivers the active ingredients more directly to the location where they are needed, as compared to a lozenge or capsule for example. The composition may be diluted to any desired concentration with the addition of water or another suitable diluent; the diluted composition may contain anywhere from about 0.1% to about 99.999% by weight water or other diluent, more preferably about 55% to about 95% water or other diluent by weight and most preferably 70% to about 90% water or other diluent by weight .

The following preferred ranges define compositions according to the invention that are suited for administration in a liquid formulation, such as a spray, according to the methods of the invention.

Each gram of the composition administered in a spray according to the methods of the present invention preferably contains about 0.001 mg to about 12 mg of a turmeric extract such as soluble or miscible oleoresin turmeric. Most preferably, each gram of the composition contains about 0.01 mg to about 9 mg of a turmeric extract such as soluble or miscible oleoresin turmeric. Each gram of the composition administered in a spray according to the methods of the present invention preferably contains about 0.001 mg to about 20 mg of a green tea extract such as green tea leaf extract. Most preferably, each gram of the composition contains about 0.01 mg to about 15 mg of a green tea extract such as green tea leaf extract.

Each gram of an optional embodiment of a composition administered in a spray according to the methods of present invention optionally contains about 0.001 mg to about 10 mg of a ginger extract such as Aquaresin® ginger. Most preferably, each gram of the composition contains about 0.01 mg to about 7 mg of a ginger extract such as Aquaresin® ginger.

Optionally, each gram of the composition also contains from about 0.0001 mg to about 0.5 mg of horseradish root extract, more preferably about 0.001 mg to about 2 mg and most preferably about 0.5 mg to about 1 mg of a horseradish root extract.

An effective amount of the composition may also be used to disinfect and/or sterilize any equipment used to administer the composition to the birds so as to inactivate some or all of any strain of the Newcastle disease virus located on the equipment. The composition may be topically applied to any equipment or machine surface to disinfect the instrument.

The invention will be further illustrated by the examples given below which are not to be construed as limiting the invention in any way. The scope of the invention is to be determined by the claims appended hereto.

### Example 1

A Suitable Formulation - Composition 1

| | Target | Target | Actual | Actual |
|---|---|---|---|---|
| Component | (wt %) | (g) | (wt g) | (wt %) |
| Turmeric | 0.6466 | 0.6466 | 0.6952 | 0.6943 |
| Oleoresin ® | | | | |
| Ginger | 0.6840 | 0.6840 | 0.6826 | 0.6817 |
| Oleoresin ® | | | | |
| Horseradish Oil | 0.063120 | 0.0631 | 0.0631 | 0.0630 |
| Green tea, | 0.4619 | 0.4619 | 0.4646 | 0.4640 |
| powered extract | | | | |
| Glycerin | 46.5723 | 46.5723 | 46.6322 | 46.5701 |
| Ethanol | 5.0000 | 5.0000 | 5.0157 | 5.0090 |
| Phospholipids | 0.5000 | 0.5000 | 0.5093 | 0.5086 |
| MCT oil | 5.0000 | 5.0000 | 5.0033 | 4.9966 |
| Water | 41.0721 | 41.0721 | 41.0673 | 41.0126 |
| Total | 99.9981 | 100.0000 | 100.1333 | 100.0000 |

This formulation may be diluted by a factor of 1-1300 with water or another suitable diluent to provide more dilute compositions for use in a variety of applications such as spraying, misting, as an aerosol or as a liquid formulation.

### Example 2

A study of the safety and tolerability of Composition 1 to chickens using various dosages and routes of administration was performed. The results demonstrated that Composition 1 is an effective and suitable liquid additive to poultry water supply, liquid additive to a nasal drop formulation or solid additive to poultry feed.

132 White Leghorn chickens, approximately 7 days old, were separated into 11 groups, administered various forms and concentrations of Composition 1 corresponding to Tables 1(a)-1(c) and subsequently exposed to the Newcastle virus.

Table 1(a)-1(c) summarize the dosage, route of administration and concentrations of a composition in accordance with the invention given to 132 White Leghorn chickens in a tolerability and toxicity study.

TABLE 1(a)

| | No. of | Chicken | | Dose/ |
|---|---|---|---|---|
| Group | Chickens | Numbers | Route of Administration | Concentration |
| 1 | 12 | 1-12 | feed (continuous) | high |
| 2 | 12 | 13-24 | feed (continuous) | med |
| 3 | 12 | 25-36 | feed (continuous) | low |
| 4 | 12 | 37-48 | water (continuous) | high |
| 5 | 12 | 49-60 | water (continuous) | med |
| 6 | 12 | 61-72 | water (continuous) | low |
| 7 | 12 | 73-84 | feed & water (continuous) | high |
| 8 | 12 | 85-96 | feed & water (continuous) | med |
| 9 | 12 | 97-108 | feed & water (continuous) | low |
| 10 | 12 | 109-120 | control (none) | — |
| 11 | 12 | 121-132 | nasal drop | — |
| Spare bird supply | 12 | n/a | n/a | — |
| Total Birds | 144 | | | |

TABLE 1(b)

| | | Dose/ | | |
|---|---|---|---|---|
| Group | Route of Administration | Concentration | Dosing Days | Dosing Duration |
| 1 | feed (continuous) | high | 1-4 | ad libitum |
| 2 | feed (continuous) | med | 1-4 | ad libitum |
| 3 | feed (continuous) | low | 1-4 | ad libitum |
| 4 | water (continuous) | high | 1-4 | ad libitum |
| 5 | water (continuous) | med | 1-4 | ad libitum |
| 6 | water (continuous) | low | 1-4 | ad libitum |
| 7 | feed & water (continuous) | high | 1-4 | ad libitum |
| 8 | feed & water (continuous) | med | 1-4 | ad libitum |
| 9 | feed & water (continuous) | low | 1-4 | ad libitum |
| 10 | control (none) | — | 1-4 | ad libitum |
| 11 | nasal drop | — | 1-4 | 1 drop per nostril 4×/day |
| Spares | n/a | — | none | none |

TABLE 1(c)

| | | | | | Total Feed | | Amount | Amount | Water | | Amount | Amount |
|---|---|---|---|---|---|---|---|---|---|---|---|---|
| | No. | | Dose/ | | Consumption | | Drug | pure | Consumption | | Drug | pure |
| | of | Route of | Con- | Days | per | % drug | Required | feed | entire study | % drug | Required | water |
| Group | Birds | Administration | centration | Dosing | study period | mixture | (gms) | required | period (ml) | mixture | (ml) | required |
| 1 | 12 | feed | high | 4 | 960 | 0.2 | 192 | 768 | 1920 | 0 | 0 | 1920 |
| 2 | 12 | feed | med | 4 | 960 | 0.12 | 115.2 | 844.8 | 1920 | 0 | 0 | 1920 |
| 3 | 12 | feed | low | 4 | 960 | 0.02 | 19.2 | 940.8 | 1920 | 0 | 0 | 1920 |
| 4 | 12 | water | high | 4 | 960 | 0 | 0 | 960 | 1920 | 0.2 | 384 | 1536 |
| 5 | 12 | water | med | 4 | 960 | 0 | 0 | 960 | 1920 | 0.12 | 230.4 | 1689.6 |
| 6 | 12 | water | low | 4 | 960 | 0 | 0 | 960 | 1920 | 0.02 | 38.4 | 1881.6 |
| 7 | 12 | feed & water | high | 4 | 960 | 0.2 | 192 | 768 | 1920 | 0.2 | 384 | 1536 |
| 8 | 12 | feed & water | med | 4 | 960 | 0.12 | 115.2 | 844.8 | 1920 | 0.12 | 230.4 | 1689.6 |
| 9 | 12 | feed & water | low | 4 | 960 | 0.02 | 19.2 | 940.8 | 1920 | 0.02 | 38.4 | 1881.6 |
| 10 | 12 | control (none) | — | 4 | 960 | 0 | 0 | 960 | 1920 | 0 | 0 | 1920 |
| 11 | 12 | nasal drop | — | 4 | 960 | 0 | 0 | 960 | — | — | 14 | — |
| Total | 132 | | | SUB- | 10560 | | 652.8 | 9907.2 | 19200 | | 1319.6 | 17894.4 |
| Birds | | | | TOTALS: | | | | | | | | |
| | | | | | 20 gms/ | | | | 40 ml/ | | | |
| | | | | | day/bird | | | | day/bird | | | |
| | | | | | | | Drug | pure | | | Drug | |
| | | | | | | | subtotal | feed | | | subtotal | pure water |
| | | | | | Total Feed | | (gms) | subtotal | Total Water | | (ml) | subtotal |
| | | | | | 10560 | | 652.8 | 9907.2 | 19200 | | 1319.6 | 17894.4 |

The chickens were housed three in each cage. The cages were maintained at about 85 degrees Fahrenheit and with a relative humidity at 65%. They were provided with 16 hours of continuous, incandescent lighting, followed by 8 hours of darkness each day.

The chickens were monitored to determine their tolerability to and the toxicity of Composition 1. Quantities of water and feed consumed by each group were assessed and individual chicken weights were routinely weighed and measured.

For chickens which were administered Composition 1 in the form of nasal drops, the nasal drops were administered, one (1) drop per each nostril, four (4) times daily, for 4 days of dosing, in alignment with the feed and water dosing groups. Drops may be administered twice in the morning with each administration being approximately 1 hour apart, and twice in the afternoon/evening also with each administration being approximately 1 hour apart. This nasal dosing schedule is flexible so that a total of 4 drops per each nostril can be administered per day, yet consecutive morning or afternoon doses are not spaced closer than 1 hour apart. Nasal drops were administered using a standard bottle type dropper containing 20 drops/ml, or 50 microliters per drop.

For chickens which were administered Composition 1 as a feed additive, the feed additive was provided for a 4 day period. One group of 24 chickens was given a high dose of the feed additive. Another group of 24 chickens were given a medium dose, and another group of 24 chickens were given a low dose. Feed and water was provided ad libitum for 4 days, under routine conditions.

For chickens which were administered Composition 1 as a water additive, the water additive was provided for a 4 day period. One group of 24 chickens were given a high dose of the water additive. Another group of 24 chickens were given a medium dose, and another group of 24 chickens were given a low dose. Water was provided to the chickens ad libitum for 4 days, under routine conditions. Feed was provided ad libitum. The control group in this experiment were housed and fed according to standard conditions for 4 days.

To determine the optimal effective dosage, each group of chickens were administered Composition 1 for a period of 1-4 days out of the week. The chickens were observed daily for any signs of general malaise or abnormal behavior, to include ruffled feathers, depressed posture, down birds, or any other indicators of stress or discomfort. Any lesions or abnormalities noted were recorded a daily basis. At the end of the study, the chickens were euthanized and individually necropsied to search for any lesions. Collected samples were placed in 10% formalin for histological assessment.

Feed and water were weighed and measured out on a per cage and per day basis. Amounts were be recorded on data collection forms. On Day 1 feed and water was measured and recorded for the first time. On Days 2-4, additional feed and water was measured and documented as added to the feed and water listing. On Day 5, the remaining feed and water were weighed, and the total feed and water consumed on a per cage basis for the entire study period was calculated.

Table 2 summarizes the change in body weight data. The differences among the treatment groups were not statistically significant for chickens which were given low feed, low water, low feed and water, medium feed, high feed, nasal and control.

TABLE 2
Body Weight (grams) Summary
Pen 18 Deleted

| Treatment, | | Pre- | Post- | Change: | |
|---|---|---|---|---|---|
| Group | Statistic | Treatment | Treatment | Post-Pre | |
| Control | N BIRDS | 9 | 9 | 9 | |
| | MEAN | 87.0 | 126.1 | 39.1 | a |
| | MEDIAN | 88.7 | 126.0 | 38.2 | |
| | STD | 5.2 | 6.4 | 2.6 | |
| | MIN | 79.8 | 118.0 | 35.7 | |
| | MAX | 94.6 | 135.0 | 43.6 | |
| | P-VALUE | | | 0.0000 | |
| High Feed | N BIRDS | 12 | 12 | 12 | |
| | MEAN | 85.8 | 122.6 | 36.8 | a |
| | MEDIAN | 85.0 | 119.5 | 36.1 | |
| | STD | 8.8 | 11.3 | 4.9 | |
| | MIN | 67.1 | 99.0 | 27.8 | |
| | MAX | 97.5 | 139.0 | 43.7 | |
| | P-VALUE | | | 0.0000 | |
| High Feed | N BIRDS | 12 | 12 | 12 | |
| and Water | MEAN | 90.5 | 88.3 | −2.1 | c |
| | MEDIAN | 89.4 | 90.7 | −0.8 | |
| | STD | 10.2 | 12.9 | 12.2 | |
| | MIN | 78.4 | 66.0 | −17.7 | |
| | MAX | 108.2 | 115.0 | 23.4 | |
| | P-VALUE | | | 0.5536 | |
| High Water | N BIRDS | 12 | 12 | 12 | |
| | MEAN | 86.5 | 93.6 | 7.1 | b |
| | MEDIAN | 87.4 | 91.3 | 10.0 | |
| | STD | 7.6 | 25.1 | 22.2 | |
| | MIN | 69.2 | 63.3 | −19.9 | |
| | MAX | 97.6 | 141.0 | 49.0 | |
| | P-VALUE | | | 0.2915 | |
| Low Feed | M BIRDS | 12 | 12 | 12 | |
| | MEAN | 92.7 | 136.7 | 44.0 | a |
| | MEDIAN | 93.5 | 135.6 | 43.5 | |
| | STD | 8.5 | 12.4 | 5.1 | |
| | MIN | 75.9 | 111.3 | 35.4 | |
| | MAX | 109.1 | 159.0 | 52.8 | |
| | P-VALUE | | | 0.0000 | |
| Low Feed, | N BIRDS | 12 | 12 | 12 | |
| and Water | MEAN | 84.4 | 121.3 | 36.9 | a |
| | MEDIAN | 81.7 | 117.9 | 35.0 | |
| | STD | 7.8 | 10.5 | 5.5 | |
| | MIN | 74.1 | 108.2 | 30.8 | |
| | MAX | 98.3 | 142.9 | 47.4 | |
| | P-VALUE | | | 0.0000 | |
| Low Water | N BIRDS | 12 | 12 | 12 | |
| | MEAN | 90.3 | 131.7 | 41.4 | a |
| | MEDIAN | 90.3 | 130.5 | 41.3 | |
| | STD | 9.1 | 13.5 | 5.5 | |
| | MIN | 74.5 | 112.7 | 32.4 | |
| | MAX | 105.2 | 160.0 | 54.8 | |
| | P-VALUE | | | 0.0000 | |
| Medium Feed | N BIRDS | 12 | 12 | 12 | |
| | MEAN | 86.8 | 127.5 | 40.7 | a |
| | MEDIAN | 88.1 | 129.4 | 40.9 | |
| | STD | 8.6 | 11.4 | 4.8 | |
| | MIN | 71.2 | 108.5 | 31.4 | |
| | MAX | 103.6 | 145.0 | 49.8 | |
| | P-VALUE | | | 0.0000 | |
| Medium Feed | N BIRDS | 12 | 12 | 1.2 | |
| and Water | MEAN | 86.5 | 78.2 | −8.4 | c |
| | MEDIAN | 86.0 | 70.4 | −14.8 | |
| | STD | B.8 | 21.4 | 13.9 | |
| | MIN | 75.8 | 58.8 | −21.3 | |
| | MAX | 101.5 | 120.4 | 18.9 | |
| | P-VALUE | | | 0.0613 | |
| Medium Water | N BIRDS | 12 | 12 | 12 | |
| | MEAN | 84.6 | 74.1 | −10.5 | c |
| | MEDIAN | 83.8 | 72.7 | −13.3 | |
| | STD | 8.0 | 9.8 | 11.5 | |
| | MIN | 74.2 | 63.0 | −22.6 | |
| | MAX | 97.3 | 92.6 | 15.5 | |
| | P-VALUE | | | 0.0089 | |
| Nasal Drop | N BIRDS | 12 | 12 | 12 | |
| | MEAN | 84.6 | 122.4 | 37.7 | a |
| | MEDIAN | 87.7 | 123.6 | 37.5 | |
| | STD | 11.5 | 16.2 | 6.1 | |
| | MIN | 64.2 | 94.6 | 30.0 | |
| | MAX | 97.7 | 146.8 | 49.1 | |
| | P-VALUE | | | 0.0000 | |

| | | |
|---|---|---|
| OVERALL P-VALUE FOR | 0.3145 | <0.0001 |
| TREATMENT GROUP | | |
| COMPARISON | | |

Table 3 summarizes the water and feed consumption data. In this table the data are summarized on a per pen basis. 4 cages that were not used were included in the analysis to show the naturally occurring feed and water loss. The differences among the treatment groups were not statistically significant for chickens which were given low feed, low water, low feed and water, medium feed, high feed, nasal and control groups.

TABLE 3
Feed and Water Consumption Summary
Pen 18 Deleted

| | | Feed | | Water | |
|---|---|---|---|---|---|
| Treatment | | Consumption | | Consumption | |
| Group | Statistic | (grams) | | (mls) | |
| CAGE | N PENS | 4 | | 4 | |
| NOT USED | MEAN | 1.8 | d | 203.8 | c |
| | MEDIAN | 1.5 | | 189.5 | |
| | STD | 1.4 | | 30.9 | |
| | MIN | 0.5 | | 186.0 | |
| | MAX | 3.7 | | 250.0 | |
| Control | N PENS | 3 | | 3 | |
| | MEAN | 194.3 | a | 434.0 | a |
| | MEDIAN | 192.1 | | 427.0 | |
| | STD | 4.3 | | 26.2 | |
| | MIN | 191.6 | | 412.0 | |
| | MAX | 199.3 | | 463.0 | |
| High F & W | N PENS | 4 | | 4 | |
| | MEAN | 78.4 | bc | 296.0 | b |
| | MEDIAN | 77.9 | | 289.5 | |
| | STD | 40.7 | | 44.7 | |
| | MIN | 38.9 | | 253.0 | |
| | MAX | 118.8 | | 352.0 | |
| High Feed | N PENS | 4 | | 4 | |
| | MEAN | 190.4 | a | 417.8 | a |
| | MEDIAN | 188.9 | | 458.0 | |
| | STD | 17.3 | | 92.2 | |
| | MIN | 172.1 | | 280.0 | |
| | MAX | 211.7 | | 475.0 | |
| High Water | N PENS | 4 | | 4 | |
| | MEAN | 97.9 | b | 262.0 | bc |
| | MEDIAN | 98.0 | | 270.5 | |
| | STD | 63.8 | | 47.6 | |
| | MIN | 27.7 | | 202.0 | |
| | MAX | 167.9 | | 305.0 | |
| Low F & W | N PENS | 4 | | 4 | |
| | MEAN | 183.0 | a | 438.8 | a |
| | MEDIAN | 178.9 | | 446.5 | |
| | STD | 9.1 | | 40.0 | |
| | MIN | 177.5 | | 387.0 | |
| | MAX | 196.5 | | 475.0 | |
| Low Feed | N PENS | 4 | | 4 | |
| | MEAN | 197.1 | a | 437.8 | a |
| | MEDIAN | 195.3 | | 441.5 | |
| | STD | 9.9 | | 21.5 | |
| | MIN | 187.0 | | 410.0 | |
| | MAX | 210.6 | | 458.0 | |
| Low Water | N PENS | 4 | | 4 | |
| | MEAN | 194.9 | a | 418.3 | a |
| | MEDIAN | 191.1 | | 421.5 | |
| | STD | 8.3 | | 48.4 | |
| | MIS | 190.0 | | 365.0 | |
| | MAX | 207.3 | | 465.0 | |
| Medium F & W | N PENS | 4 | | 4 | |
| | MEAN | 49.6 | c | 277.0 | b |
| | MEDIAN | 47.8 | | 284.5 | |
| | STD | 16.3 | | 39.1 | |
| | MIN | 33.7 | | 228.0 | |
| | MAX | 69.1 | | 311.0 | |
| Medium Feed | N PENS | 4 | | 4 | |
| | MEAN | 191.8 a | | 447.3 | a |
| | MEDIAN | 197.3 | | 457.0 | |
| | STD | 18.2 | | 32.7 | |
| | MIN | 165.8 | | 400.0 | |
| | MAX | 206.8 | | 475.0 | |
| Medium Water | N PENS | 4 | | 4 | |
| | MEAN | 51.9 | c | 262.3 | bc |
| | MEDIAN | 52.8 | | 271.5 | |
| | STD | 7.0 | | 23.0 | |
| | MIN | 42.9 | | 220.0 | |
| | MAX | 59.3 | | 286.0 | |
| Nasal Drops | N PENS | 4 | | 4 | |
| | MEAN | 181.6 | a | 427.5 | a |
| | MEDIAN | 186.5 | | 435.5 | |
| | STD | 12.1 | | 36.1 | |
| | MIN | 163.8 | | 382.0 | |
| | MAX | 189.4 | | 457.0 | |

| | | |
|---|---|---|
| OVERALL P-VALUE FOR | <0.0001 | <0.0001 |
| TREATMENT GROUP | | |
| COMPARISON | | |

Table 4 summarizes the ratio of weight gain to feed consumption data. In this table, the data are summarized on a per pen basis. The analysis revealed that the differences among the following treatment groups were not statistically significant: low feed, low water, low feed and water, medium feed, high feed, nasal and control.

TABLE 4
Body Weight (grams)/Feed Consumption Summary (grams)
Pen 18 Deleted

| | | Body | |
|---|---|---|---|
| Treatment | | Weight/Feed | |
| Group | Statistic | Consumption | |
| Control | N PENS | 3 | |
| | MEAN | 0.603 | a |
| | MEDIAN | 0.600 | |
| | STD | 0.016 | |
| | MIN | 0.589 | |
| | MAX | 0.620 | |
| | P-VALUE | 0.0002 | |
| High F & W | N PENS | 4 | |
| | MEAN | −0.066 | ab |
| | MEDIAN | −0.159 | |
| | STD | 0.568 | |
| | MIN | −0.638 | |
| | MAX | 0.690 | |
| | P-VALUE | 0.8303 | |
| High Feed | N PENS | 4 | |
| | MEAN | 0.582 | a |
| | MEDIAN | 0.582 | |
| | STD | 0.056 | |
| | MIN | 0.523 | |
| | MAX | 0.640 | |
| | P-VALUE | 0.0002 | |
| High Water | N PENS | 4 | |
| | MEAN | −0.254 | b |
| | MEDIAN | 0.199 | |
| | STD | 1.181 | |
| | MIN | −2.004 | |
| | MAX | 0.590 | |
| | P-VALUE | 0.6963 | |
| Low F & W | N PENS | 4 | |
| | MEAN | 0.604 | a |
| | MEDIAN | 0.622 | |
| | STD | 0.044 | |
| | MIN | 0.539 | |
| | MAX | 0.633 | |
| | P-VALUE | 0.0001 | |
| Low Feed | N PENS | 4 | |
| | MEAN | 0.669 | a |
| | MEDIAN | 0.670 | |
| | STD | 0.025 | |
| | MIN | 0.645 | |
| | MAX | 0.691 | |
| | P-VALUE | 0.0000 | |
| Low Water | N PENS | 4 | |
| | MEAN | 0.638 | a |
| | MEDIAN | 0.638 | |
| | STD | 0.008 | |
| | MIN | 0.627 | |
| | MAX | 0.646 | |
| | P-VALUE | 0.0000 | |
| Medium F & W | N PENS | 4 | |
| | MEAN | −0.708 | b |
| | MEDIAN | −0.778 | |
| | STD | 0.843 | |
| | MIN | −1.474 | |
| | MAX | 0.197 | |
| | P-VALUE | 0.1916 | |
| Medium Feed | N PENS | 4 | |
| | MEAN | 0.636 | a |
| | MEDIAN | 0.630 | |
| | STD | 0.027 | |
| | MIN | 0.610 | |
| | MAX | 0.674 | |
| | P-VALUE | 0.0000 | |
| Medium Water | N PENS | 4 | |
| | MEAN | −0.659 | b |
| | MEDIAN | −0.509 | |
| | STD | 0.556 | |
| | MIN | −1.415 | |
| | MAX | −0.203 | |
| | P-VALUE | 0.0985 | |
| Nasal Drops | N PENS | 4 | |
| | MEAN | 0.623 | a |
| | MEDIAN | 0.629 | |
| | STD | 0.015 | |
| | MIN | 0.600 | |
| | MAX | 0.634 | |
| | P-VALUE | 0.0000 | |

These results provide suitable dosing for Composition 1 for poultry. The data demonstrates that Composition 1 can be provided to growing poultry in a medicated feed at various (low, medium, and high) concentrations, without safety issues or tolerability problems.

### Example 3

The undiluted antiviral composition of Example 1, Composition diluted in 10-fold serial dilutions were prepared and tested for antiviral activity against NDV in VERO E6 cells and in 10-day old embryonating chicken eggs. In addition, a placebo was similarly diluted and tested.

The continuous cell line VERO E6 (CRL-1586) was obtained from the American Type Culture Collection (Rockdale, Md.) and propagated in Minimum Essential Medium (Eagle) with 2 mM L-glutamine, 1.5 g/L sodium bicarbonate, 0.1 mM non-essential amino acids, 1.0 mM sodium pyruvate, and 10% fetal bovine serum (Invitrogen Corp [Gibco], Carlsbad, Calif.) at 37 C and 5% CO₂. The cells were grown in a T75 flask (BD Biosciences, Franklin Lakes, N.J.) and transferred to 96 well plates and grown to 90% confluence.

A 1×10³ concentration of B1/B1 strain of NDV, a tissue culture infectious dose₅₀ (TCID₅₀), was mixed with seven 10-fold serial dilutions (beginning with a dilution of 1×10⁻³, which is nontoxic for the cells) of the antiviral compound or the placebo prepared in cell culture maintenance media (containing 1% fetal bovine serum). The mixtures were incubated at room temperature for 30 minutes; then nine 10-fold serial dilutions of each mixture were prepared for inoculation onto cells. The cell culture media was removed from the cells and the mixtures were inoculated onto the monolayers. Negative control wells receiving cell culture maintenance media only were also included in the experiment. The cells were incubated for 7 days at 37 C and 5% CO₂ and examined twice daily for cytopathic effects (CPE).

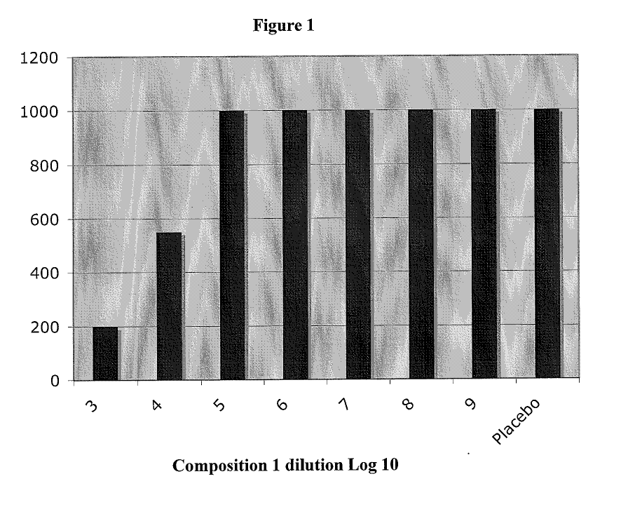

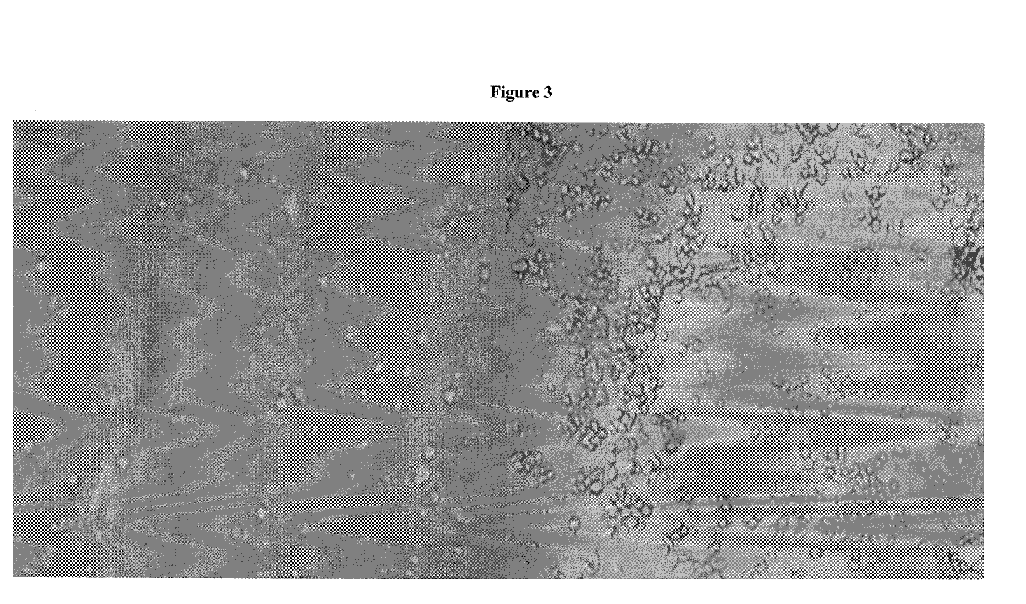

FIG. 1 shows the results of the antiviral affect on NDV. FIG. 3 shows VERO E6 cells protected from viral infection by the composition of the present invention and cells, to which the composition had not been applied, infected with NDV viral CPE. A dilution of 1×10⁻³ reduced the TCID₅₀ titer of NDV 5-fold and a 1×10⁻⁴ dilution of the composition reduced the NDV titer 1.8-fold. None of the higher dilutions of the composition reduced the titer of NDV. By comparison, no reduction in virus titer for NDV was observed for the placebo at any dilution, which suggests that the composition was responsible for the antiviral effect. Additionally, none of the negative control wells had CPE.

Embryonic chicken eggs, infected with NDV, were also tested to determine the efficacy of the composition. The embryonic chicken eggs were prepared in the same fashion as that of the VERO E6 cells. The experimental design was the same, except 10-day old embryonating chicken eggs were inoculated instead of tissue culture cells and the beginning concentration of Composition 3 and the placebo was undiluted since the compounds are not toxic for the embryos. Specific pathogen free (SPF) fertile chicken eggs were obtained from Sunrise farms (Catskill, N.Y.) and incubated at 37 C for 10 days. The embryonated eggs were inoculated into the chorioallantoic sac (CAS) with 200 ul of undiluted and each of the 10-fold dilutions of the composition or the placebo prepared in PBS (pH 7.4) and mixed with either 1×10⁴ embryo infectious dose₅₀ (EID₅₀) of 1×10⁷ EID₅₀ of NDV. Negative control eggs that received PBS only were also included. The eggs were incubated at 37 C and candled daily for 7 days to record mortality. Any mortality occurring within the first 24 hours was considered to be due to trauma associated with inoculation and disregarded. On the 7^{th} day, all the remaining eggs were chilled to 4 C and opened to examine the embryos for clinical signs.

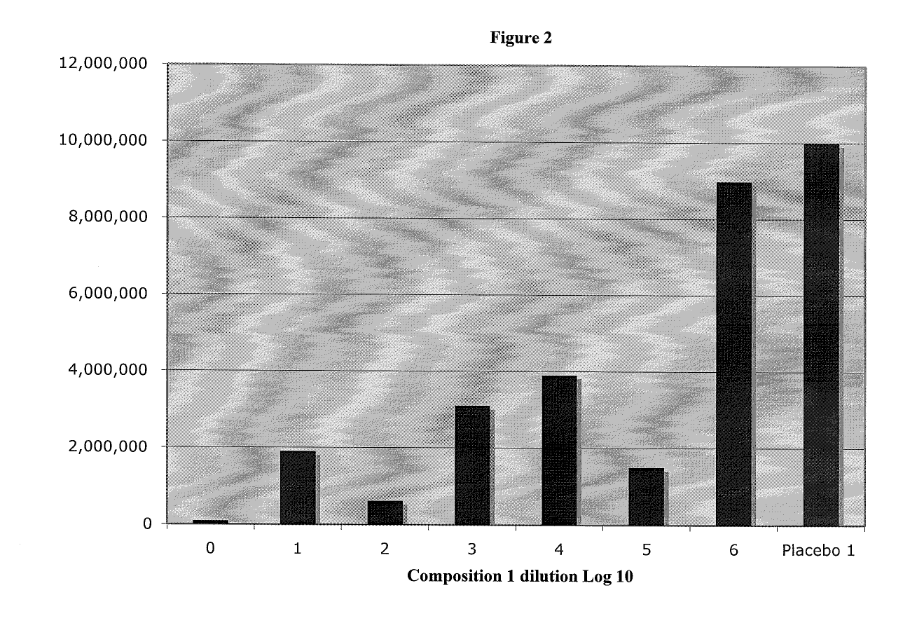

FIG. 2 shows a 100-fold reduction in titer with the undiluted composition whereas less than a 100-fold reduction was observed for dilutions of the composition from 1×10⁻¹ to 1×10⁻⁵ and only a slight reduction was observed at a 1×10⁻⁶ dilution of the composition. Similar to the VERO E6 cells, the placebo did not reduce the titer of either virus in embryonating eggs, indicating that the active ingredient in the composition was responsible for the antiviral effect. Thus, Composition 1 appears to have measurable affects against NDV at low and high concentrations.

It is to be understood, however, that even though numerous characteristics and advantages of the present invention have been set forth in the foregoing description, together with details of the structure and function of the invention, the disclosure is illustrative only, and changes may be made in detail, especially in matters of shape, size and arrangement of parts within the principles of the invention to the full extent indicated by the broad general meaning of the terms in which the appended claims are expressed.

1. A method for treatment of a bird having a Newcastle disease viral strain, comprising the step of administering to a bird that has a Newcastle disease viral strain, a safe and effective amount of composition comprising:
a first ingredient selected from the group consisting of turmeric powder extract, turmeric fluid extract, turmeric extract, turmeric powder, at least a part of a whole plant of turmeric, a turmeric tincture, and mixtures thereof,
a second ingredient selected from the group consisting of green tea powder, green tea powder extract, green tea fluid extract, at least a part of a whole plant of green tea, tinctures of green tea and mixtures thereof;
a third ingredient selected from the group consisting of ginger powder extract, ginger fluid extract, ginger powder, at least a part of a whole plant of ginger, a ginger tincture and mixtures thereof; and
an acceptable carrier;
said amount being effective, when administered, to reduce an incidence of other birds contracting Newcastle disease.

2. The method of claim 1, wherein the composition is administered in a form selected from a group consisting of a dry formulation, a liquid formulation, a tablet, a capsule, feed additive and water additive.

3. The method of claim 1, wherein the composition is administered as an aerosol, a spray or a mist.

4. The method of claim 1, wherein the composition is administered in ovo.

5. The method of claim 1, wherein administering the composition further comprises the step of using an effective amount of the composition to disinfect an item of equipment so as to render inactive at least some Newcastle disease virus located on the equipment.

6. (canceled)

7. The method of claim 1, wherein the first ingredient comprises turmeric extract and the second ingredient comprises green tea extract.

8. The method of claim 1, wherein each gram of the composition contains about 0.001 mg to about 20 mg of green tea extract, and about 0.001 mg to about 30 mg of turmeric powder extract.

9-10. (canceled)

11. The method of claim 7, wherein the third ingredient comprises ginger extract.

12. The method of claim 11, wherein the composition contains about 0.001 mg to about 30 mg of ginger extract.

13. The method of claim 1, wherein the composition further comprises a fourth ingredient selected from the group consisting of horseradish oil, horseradish powder extracts, horseradish fluid extracts and horseradish root extracts.

14. The method of claim 1, wherein the composition further comprises one or more ingredients selected from the group consisting of ethanol, propylene glycol, glycerine, phospholipids, medium chain triglyceride oil and water.

15-26. (canceled)

27. The method of claim 13, wherein the composition comprises horseradish oil.

28-43. (canceled)

44. A method for the prophylactic use of a composition to reduce an incidence or transmissivity of Newcastle disease, comprising the step of administering to a bird that has been, or will be, exposed to Newcastle disease, a safe and effective amount of a composition comprising:
a first ingredient selected from a group consisting of turmeric powder extract, turmeric fluid extract, turmeric extract, turmeric powder, at least a part of a whole plant of turmeric, a turmeric tincture, and mixtures thereof;
a second ingredient selected from the group consisting of green tea powder, green tea powder extract, green tea fluid extract, at least a part of a whole plant of green tea, tinctures of green tea and mixtures thereof;
a third ingredient selected from the group consisting of ginger powder extract, ginger fluid extract, ginger powder, at least a part of a whole plant of ginger, a ginger tincture and mixtures thereof; and
an acceptable carrier;
said amount being effective, when administered, to reduce the incidence of Newcastle disease in said bird or to reduce an incidence of Newcastle disease in other birds exposed to said treated bird.

45. The method of claim 44, wherein the composition is administered in a form selected from a group consisting of a dry formulation, a liquid formulation, a tablet, a capsule, feed additive and water additive.

46. The method of claim 44, wherein the composition is administered as an aerosol, a spray or a mist.

47. The method of claim 44, wherein the composition is administered in ovo.

48. The method of claim 44, wherein administering the composition further comprises the step of using an effective amount of the composition to disinfect an item of equipment so as to render inactive at least some Newcastle disease virus located on the equipment.

49. The method of claim 44, wherein the first ingredient comprises turmeric extract and the second ingredient comprises green tea extract.

50. The method of claim 44, wherein each gram of the composition contains about 0.001 mg to about 20 mg of green tea extract, and about 0.001 mg to about 30 mg of turmeric powder extract.

51. The method of claim 49, wherein the third ingredient comprises ginger extract.

52. The method of claim 51, wherein the composition contains about 0.001 mg to about 30 mg of ginger extract.

53. The method of claim 44, wherein the composition further comprises a fourth ingredient selected from the group consisting of horseradish oil, horseradish powder extracts, horseradish fluid extracts and horseradish root extracts.

54. The method of claim 44, wherein the composition further comprises one or more ingredients selected from the group consisting of ethanol, propylene glycol, glycerine, phospholipids, medium chain triglyceride oil and water.

55. The method of claim 53, wherein the composition comprises horseradish oil.
